(19) United States
(12) Patent Application Publication
(10) Pub. No.: US 2023/0215844 A1
(43) Pub. Date: Jul. 6, 2023

(54) SENSING DEVICE AND MANUFACTURING METHOD THEREOF

(71) Applicant: FOSHAN NATIONSTAR OPTOELECTRONICS CO., LTD., Foshan (CN)

(72) Inventors: Junzheng Li, Foshan (CN); Mingjun Zhu, Foshan (CN); Meiqin Lei, Foshan (CN); Yan Chen, Foshan (CN); Jundong Qiu, Foshan (CN); Huijuan Liu, Foshan (CN); Yinling Zheng, Foshan (CN)

(73) Assignee: FOSHAN NATIONSTAR OPTOELECTRONICS CO., LTD., Foshan, GD (CN)

(21) Appl. No.: 17/892,298

(22) Filed: Aug. 22, 2022

(30) Foreign Application Priority Data
Dec. 31, 2021 (CN) 20111676587.4
Dec. 31, 2021 (CN) 20211167276.X

(51) Int. Cl. H01L 25/075; H01L 33/52; H01L 33/60; H01L 33/62

(52) U.S. Cl. CPC H01L 25/0753; H01L 33/52; H01L 33/60; H01L 33/62; H01L 2933/0066; A61B 5/14552

(57) ABSTRACT

Provided are a sensing device and a manufacturing method thereof. The sensing device includes a substrate, a red light chip, an infrared light chip, and a green light chip. The red light chip, the infrared light chip, and the green light chip are disposed on the front face of the substrate. Five front face pads are disposed on the front face of the substrate. Five back face pads are disposed on the back face of the substrate. The third back face pad is connected to the fourth back face pad by a conductive line. One of the five front face pads is electrically connected to a corresponding one of the five back face pads.

### CROSS-REFERENCE TO RELATED APPLICATION(S)

This application claims priority to Chinese Patent Application No. 202111677276.X filed Dec. 31, 2021 and Chinese Patent Application No. 202111676587.4 filed Dec. 31, 2021, the disclosures of which are incorporated herein by reference in their entireties.

### TECHNICAL FIELD

Embodiments of the present disclosure relate to the technology of intelligent wearable devices and, in particular, to a sensing device and a manufacturing method thereof.

### BACKGROUND

The existing intelligent wearable devices are equipped with sensing devices to monitor the blood oxygen and heart rate of users and provide health monitoring. Therefore, multiple light-emitting chips need to be installed in the sensing devices. However, the light emitted by the multiple light-emitting chips is scattered, affecting the monitoring effect. Moreover, the sensing devices heat seriously after working for a long time, affecting the service life of the devices. However, the heat dissipation effect of the existing sensing devices cannot satisfy the long-term working of multiple light-emitting chips.

### SUMMARY

Embodiments of the present disclosure provide a sensing device and a manufacturing method thereof.

In a first aspect, an embodiment of the present disclosure provides a sensing device. The sensing device includes a substrate, a red light chip, an infrared light chip, and a green light chip. The red light chip, the infrared light chip, and the green light chip are disposed on a front face of the substrate.

Five front face pads are disposed on the front face of the substrate. The five front face pads include a first front face pad, a second front face pad, a third front face pad, a fourth front face pad, and a fifth front face pad.

Five back face pads are disposed on a back face of the substrate. The five back face pads include a first back face pad, a second back face pad, a third back face pad, a fourth back face pad, and a fifth back face pad. The third back face pad is connected to the fourth back face pad by a conductive line.

One of the five front face pads is electrically connected to a corresponding one of the five back face pads.

The red light chip, the infrared light chip, and the green light chip are electrically connected to the five front face pads.

In an embodiment, a bottom electrode of the green light chip is bonded on the second front face pad, and a top electrode of the green light chip is electrically connected to the third front face pad by a metal wire; a bottom electrode of the red light chip is bonded on the fourth front face pad, and a top electrode of the red light chip is electrically connected to the fifth front face pad by a metal wire; a bottom electrode of the infrared light chip is bonded on the fourth front face pad, and a top electrode of the infrared light chip is electrically connected to the first front face pad by a metal wire.

In an embodiment, each of the size of the red light chip and the size of the infrared light chip is less than the size of the green light chip.

In an embodiment, the red light chip, the infrared light chip, and the green light chip are disposed on the front face of the substrate in a shape of a triangle. The red light chip and the infrared light chip are disposed on the same side. The green light chip is disposed on another side.

In an embodiment, the front face of the substrate is provided with an encapsulation layer formed by an encapsulation material. The encapsulation layer is surrounded by a white blocking wall.

In an embodiment, a light-emitting angle of the sensing device is α. The constraint relationship of α is 120°≤α≤130°.

In an embodiment, a sixth front face pad is further disposed on the front face of the substrate, and/or a sixth back face pad is further disposed on the back face of the substrate, where the sixth front face pad is an idle front face pad, and the sixth back face pad is an idle back face pad.

In an embodiment, the first back face pad, the second back face pad, the third back face pad, the fourth back face pad, the fifth back face pad, and the sixth back face pad are separated in two columns and disposed on the back face of the substrate; and the third back face pad and the fourth back face pad are disposed at diagonal positions.

In an embodiment, the third back face pad and the fourth back face pad are common-positive back face pads; the first back face pad, the second back face pad, and the fifth back face pad are negative back face pads; and the sixth back face pad is the idle back face pad.

Alternatively, the third back face pad and the fourth back face pad are common-negative back face pads; the first back face pad, the second back face pad, and the fifth back face pad are positive back face pads; and the sixth back face pad is the idle back face pad.

In an embodiment, the conductive line includes three segments, and an included angle between two adjacent segments of the three segments is a right angle.

In an embodiment, the back face of the substrate is coated with green oil; the middle segment of the three segments is close to one column of the two columns in which the first back face pad, the second back face pad, the third back face pad, the fourth back face pad, the fifth back face pad, and the sixth back face pad are disposed; a position between the middle segment of the three segments and another one column of the two columns in which the first back face pad, the second back face pad, the third back face pad, the fourth back face pad, the fifth back face pad, and the sixth back face pad are disposed is blank to form an electrical mark.

In an embodiment, the thickness of the conductive line is less than the thickness of one of the first back face pad, the second back face pad, the third back face pad, the fourth back face pad, the fifth back face pad, or the sixth back face pad.

In an embodiment, the sensing device further includes a metal wire and an encapsulation layer. The red light chip, the infrared light chip, and the green light chip are light-emitting diode (LED) chips. Each of at least one LED chip has a top electrode. The top electrode is electrically connected to one front face pad by the metal wire. The LED chip and the metal wire are encapsulated by the encapsulation layer. The metal wire includes a first segment, a second segment, and a third segment connected in sequence. The head end of the first segment is bonded on the front face pad. The included angle, facing away from the LED chip, between the first segment and the top face of the front face pads is a. The transition between the first segment and the second segment is a rounded corner. The included angle, facing the LED chip, between the first segment and the second segment is b. The connection position between the second segment and the third segment is located directly above the edge of the top face of the LED chips. The tail end of the third segment is bonded on the top electrode. The included angle, facing the LED chip, between the third segment and the top face of the top electrode is c.

In an embodiment, the middle portion of the first segment has a first camber arch facing away from the LED chip.

In an embodiment, the middle portion of the second segment has a second camber arch facing a downward direction.

In an embodiment, the height of the LED chip is h, the height of the highest point of the metal wire is less than or equal to 1.5 h, and the height of the highest point of the metal wire is greater than h.

In an embodiment, the distance between the connection position between the second segment and the third segment and the top face of the LED chip is greater than the wire diameter of the metal wire.

In an embodiment, the height of the LED chip is h, the distance between the connection position between the second segment and the third segment and the top face of the LED chip is less than or equal to h/2, and the distance between the connection position between the second segment and the third segment to the top face of the LED chip is greater than or equal to h/6.

In an embodiment, the value range of the rounded corner of the connection position between the first segment and the second segment is [80°, 100° ], and the distance between the LED chip and the front face pad is 90 μm to 1050 μm. The value range of the included angle a is 80°<a<110°. The value range of the included angle c is 8°<c<25°. The width of the LED chip is 2 to 2.5 times the distance between an electrode of the LED chip and the edge of the LED chip. The value range of the included angle d between the second segment and the horizontal plane is 8°<d<25°.

In a second aspect, an embodiment of the present disclosure provides a manufacturing method of a sensing device. The method is configured to manufacture the sensing device in the first aspect.

### BRIEF DESCRIPTION OF DRAWINGS

FIG. 1 is a diagram illustrating the structure of a sensing device according to an embodiment of the present disclosure;

FIG. 2 is a diagram illustrating the structure of a front face of a substrate according to an embodiment of the present disclosure;

FIG. 3 is a diagram illustrating the structure of a back face of a substrate according to an embodiment of the present disclosure;

FIG. 4 is a diagram illustrating the structure of a side face of a sensing device according to an embodiment of the present disclosure;

FIG. 5 is a flowchart of a manufacturing method of a sensing device according to an embodiment of the present disclosure;

FIG. 6 is a sectional view of a sensing device according to an embodiment of the present disclosure; and

FIG. 7 is a flowchart of a wire bonding method according to an embodiment of the present disclosure.

### DETAILED DESCRIPTION

The present disclosure is further described hereinafter in detail in conjunction with drawings and embodiments. It is to be understood that the embodiments described herein are intended to explain the present disclosure and not to limit the present disclosure. Additionally, it is to be noted that for ease of description, only part, not all, of the structures related to the present disclosure are illustrated in the drawings.

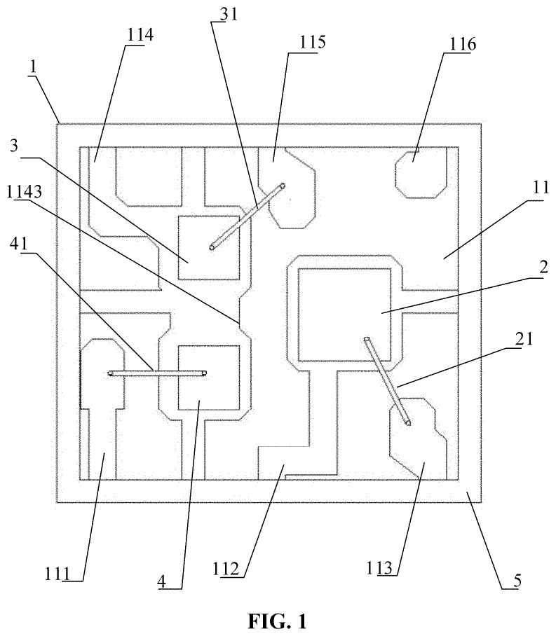

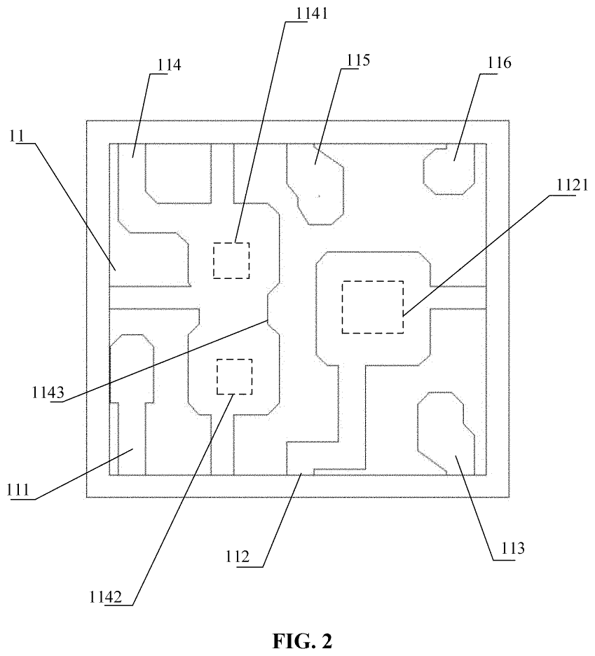

FIG. 1 is a diagram illustrating the structure of a sensing device according to an embodiment of the present disclosure. FIG. 2 is a diagram illustrating the structure of a front face of a substrate according to an embodiment of the present disclosure. Referring to FIG. 1 and FIG. 2, a sensing device includes a substrate 1, a green light chip 2, a red light chip 3, and an infrared light chip 4. The green light chip 2, the red light chip 3, and the infrared light chip 4 are arranged on the substrate 1 in a shape of a triangle. A first front face pad 111, a second front face pad 112, a third front face pad 113, a fourth front face pad 114, a fifth front face pad 115, and a sixth front face pad 116 are disposed on the substrate 1. A first chip slot 1141 and a second chip slot 1142 are disposed on the fourth front face pad 114. A third chip slot 1121 is disposed on the second front face pad 112. The red light chip 3 is bonded on the first chip slot 1141. The infrared light chip 4 is bonded on the second chip slot 1142. The green light chip 2 is bonded on the third chip slot 1121.

Further, the fourth front face pad 114 is provided with a groove portion 1143. The groove portion 1143 is disposed between the first chip slot 1141 and the second chip slot 1142 to facilitate the identification of die bonding. When the die bonding operation is performed, the positions of the first chip slot 1141 and the second chip slot 1142 can be accurately identified, thereby improving the reliability of the device.

Further, the red light chip 3 and the infrared light chip 4 are vertical structure chips. The vertical structure has the advantages of high brightness, low voltage drop, and low power consumption. The green light chip 2 may be a vertical structure chip or a lateral structure chip.

In an embodiment, the red light chip 3 is fixed to the first chip slot 1141, and the infrared light chip 4 is fixed to the second chip slot 1142. That is, the red light chip 3 and the infrared light chip 4 are arranged on the fourth front face pad 114. The green light chip 2 is fixed to the third chip slot 1121, that is, the green light chip 2 is arranged on the second front face pad 112. Further, the red light chip 3 is connected to the fifth front face pad 115 by a first metal wire 31. The infrared light chip 4 is connected to the first front face pad 111 by a second metal wire 41. The green light chip 2 is connected to the third front face pad 113 by a third metal wire 21.

In an embodiment, the green light chip 2 emits green light. A receiving device corresponding to the sensing device receives the green light reflected from a human body to test the heart rate. The red light chip 3 emits red light. The receiving device corresponding to the sensing device receives the red light reflected from the human body to test the hemoglobin concentration. The infrared light chip 4 emits infrared light. The receiving device corresponding to the sensing device receives the infrared light reflected from the human body to detect the blood oxygen saturation. The sensing device monitors the blood oxygen and the heart rate of the human body through the green light chip 2, the red light chip 3, and the infrared light chip 4.

Further, the size of the green light chip 2 is greater than the size of the red light chip 3 or the size of the infrared light chip 4 so that the green light of high brightness can be emitted. Therefore, the brightness requirement of the receiving device for the reflected green light can be met, and the heart rate of the human body can be detected.

In an embodiment, the light-emitting angle of the sensing device is α. The constraint relationship of α is 120°≤α≤130°, satisfying human body detection requirements of the sensing device. Thus, the receiving device can receive enough reflected light with less interference light, and the detection results are accurate.

Further, the sensing device is encapsulated by an encapsulation material through a secondary encapsulation process, and an encapsulation layer 10 is formed. The encapsulation layer 10 on the front face 11 of the substrate is surrounded by a white blocking wall 5. A transition connection with an arc is disposed at the connection between the white blocking wall 5 and the substrate 1. The white blocking wall 5 reduces absorption and refraction of light emitted from the green light chip 2, the red light chip 3, and the infrared light chip 4 and increases the light emission intensity of the green light chip 2, the red light chip 3, and the infrared light chip 4.

Further, the height of the chip on the sensing device is h, and the height of the encapsulation layer 10 is H. The constraint relationship between H and h is 1.5 h<H<4 h. The value range of H is 200 μm<H<600 μm.

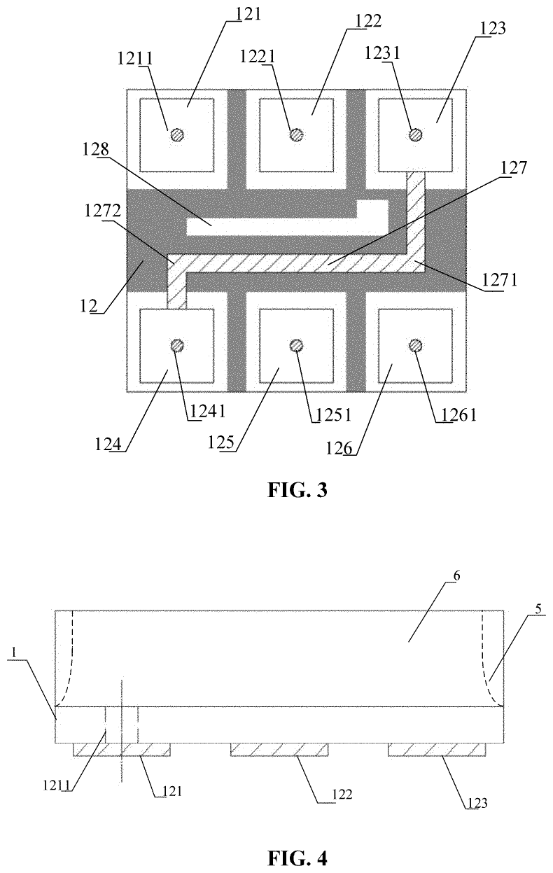

In an embodiment, FIG. 3 is a diagram illustrating the structure of a back face of a substrate according to an embodiment of the present disclosure. Referring to FIG. 3, six back face pads are disposed on a back face 12 of the substrate 1. The six back face pads include a first back face pad 121, a second back face pad 122, a third back face pad 123, a fourth back face pad 124, a fifth back face pad 125, and a sixth back face pad 126. The first back face pad 121, the second back face pad 122, the third back face pad 123, the fourth back face pad 124, the fifth back face pad 125, and the sixth back face pad 126 correspond to the first front face pad 111, the second front face pad 112, the third front face pad 113, the fourth front face pad 114, the fifth front face pad 115, and the sixth front face pad 116, respectively.

The first back face pad 121, the second back face pad 122, the third back face pad 123, the fourth back face pad 124, the fifth back face pad 125, and the sixth back face pad 126 are separated into two columns and disposed on two sides of the back face 12 of the substrate. Further, the third back face pad 123 and the fourth back face pad 124 are disposed at diagonal positions.

In an embodiment, the six back face pads are independent of each other. During the processing and production process, back face pads of two adjacent devices are not connected to each other. That is, the six back face pads are located in the outer edge of the substrate 1 and are not connected to the outer edge of the substrate 1, facilitating the cutting and separation into separate sensing devices and avoiding burrs on sidewalls of the devices.

It is to be noted that, to facilitate processing and production, corresponding front face pads on two connected sides between any two adjacent devices are connected. After the separate devices are obtained by devision, copper foils of the six front face pads are exposed on the corresponding sidewalls of the substrate 1.

In an embodiment, six metal guide pillars are disposed on the six back face pads. The six metal guide pillars include a first metal guide pillar 1211, a second metal guide pillar 1221, a third metal guide pillar 1231, a fourth metal guide pillar 1241, a fifth metal guide pillar 1251, and a sixth metal guide pillar 1261. The first metal guide pillar 1211, the second metal guide pillar 1221, the third metal guide pillar 1231, the fourth metal guide pillar 1241, the fifth metal guide pillar 1251, and the sixth metal guide pillar 1261 are configured to connect the six front face pads on the front face 11 of the substrate 1.

Further, the six metal guide pillars are disposed at any position on the projection of the six back face pads.

In an embodiment, the six back face pads may be correspondingly connected to the six front face pads by ink plug holes, and the six back face pads may be correspondingly connected to the six front face pads by resin plug holes.

In an embodiment, FIG. 4 is a diagram illustrating the structure of a side face of a sensing device according to an embodiment of the present disclosure. Referring to FIG. 4, the first metal guide pillar 1211 penetrates through the substrate 1. The first front face pad 111 and the first back face pad 121 are connected and conducted by the first metal guide pillar 1211.

The second front face pad 112 and the second back face pad 122, the third front face pad 113 and the third back face pad 123, the fourth front face pad 114 and the fourth back face pad 124, the fifth front face pad 115 and the fifth back face pad 125, the sixth front face pad 116 and the sixth back face pad 126 have the same metal guide pillar structure as that of the first front face pad 111 and the first back face pad 121. For specific structural features and functional roles, reference may be made to the structural features and functional roles of the metal guide pillar between the first front face pad 111 and the first back face pad 121, and details are not described herein.

Further, the red light chip 3 and the infrared light chip 4 are connected to the fourth back face pad 124 by the fourth front face pad 114. The red light chip 3 is connected to the fifth back face pad 125 by the fifth front face pad 115. The infrared light chip 4 is connected to the first back face pad 121 by the first front face pad 111. The green light chip 2 is connected to the second back face pad 122 by the second front face pad 112. The green light chip 2 is connected to the third back face pad 123 by the third front face pad 113. The green light chip 2, the red light chip 3, and the infrared light chip 4 can work normally.

In an embodiment, the first chip slot 1141 and the second chip slot 1142 may be disposed on the third front face pad 113, and the third chip slot 1121 may be disposed on the fourth front face pad 114. Alternatively, the first chip slot 1141 and the second chip slot 1142 may be disposed on the fourth front face pad 114, and the third chip slot 1121 may be disposed on the third front face pad 113.

In an embodiment, among the first back face pad 121, the second back face pad 122, the third back face pad 123, the fourth back face pad 124, the fifth back face pad 125, and the sixth back face pad 126 disposed on the back face 12 of the substrate, the third back face pad 123 and the fourth back face pad 124 are back face pads having the commonality. Further, the third back face pad 123 and the fourth back face pad 124 are common-positive back face pads. The first back face pad 121, the second back face pad 122, and the fifth back face pad 125 are negative back face pads. The sixth back face pad 126 is an idle back face pad.

In an embodiment, the third back face pad 123 and the fourth back face pad 124 are common-negative back face pads. The first back face pad 121, the second back face pad 122 and the fifth back face pad 125 are positive back face pads. The sixth back face pad 126 is an idle back face pad.

Further, the first back face pad 121, the second back face pad 122, the third back face pad 123, the fourth back face pad 124, the fifth back face pad 125, and the sixth back face pad 126 are disposed on the back face 12 of the substrate to facilitate heat dissipation.

In an embodiment, the third back face pad 123 and the fourth back face pad 124 are common-positive back face pads, and the driving design cost of the sensing device is low.

Further, the sixth back face pad 126 is an idle back face pad. The sixth back face pad 126 has no electrical properties. The first back face pad 121, the second back face pad 122, the third back face pad 123, the fourth back face pad 124, the fifth back face pad 125, and the sixth back face pad 126 are disposed to achieve symmetry of the back face pads. The sixth back face pad 126 is used for maintaining stability of the substrate 1 when tin is soldered to the device and maintaining the substrate 1 in a horizontal state.

In an embodiment, the first back face pad 121, the second back face pad 122, the third back face pad 123, the fourth back face pad 124, the fifth back face pad 125, and the sixth back face pad 126 are symmetrically distributed on the back face 12 of the substrate. The first back face pad 121, the second back face pad 122, and the third back face pad 123 are arranged in a column on one side of the back face 12 of the substrate. The fourth back face pad 124, the fifth back face pad 125, and the sixth back face pad 126 are arranged in a column on another side of the back face 12 of the substrate. The third back face pad 123 and the fourth back face pad 124 are disposed at diagonal positions of the back face 12 of the substrate. The third back face pad 123 is connected to the fourth back face pad 124 by a conductive line 127. The third back face pad 123 and the fourth back face pad 124 are disposed on a diagonal line so that the formation of the interleaved wires by the external circuit connection wires of the sensing device can be reduced and avoided, and the complexity of the overall circuit can be reduced.

The back face 12 of the substrate is coated with green oil. The first back face pad 121, the second back face pad 122, the third back face pad 123, the fourth back face pad 124, the fifth back face pad 125, and the sixth back face pad 126 are blocked by the green oil to avoid the problem that when the sensing device is soldered, the boundary of the line is broken due to the connection of the back face pads.

Further, a position close to the middle of the back face 12 of the substrate is left blank to form an electrical mark 128. The electrical mark 128 is used for marking the polarity of the first back face pad 121, the second back face pad 122, the third back face pad 123, the fourth back face pad 124, the fifth back face pad 125, and the sixth back face pad 126. The conductive line 127 is divided into three segments. The three segments are connected by a first right-angle turning portion 1271 and a second right-angle turning portion 1272. The middle segment of the three segments is close to one column back face pads of two columns of the back face pads. The first right-angle turning portion 1271 and the second right-angle turning portion 1272 increase the connection distance between the third back face pad 123 and the fourth back face pad 124 and ensure that the third back face pad 123 and the fourth back face pad 124 can be safely connected. Further, the distance between the middle segment of the conductive line 127 and another column back face pads among the first back face pad 121, the second back face pad 122, the third back face pad 123, the fourth back face pad 124, the fifth back face pad 125, and the sixth back face pad 126 ensures sufficient space for the blank to form the electrical mark 128.

In an embodiment, the thickness of the conductive line 127 is less than the thickness of any one of the first back face pad 121, the second back face pad 122, the third back face pad 123, the fourth back face pad 124, the fifth back face pad 125, or the sixth back face pad 126 to prevent instability of the device from being lifted up during the soldering process of the device, thereby affecting the use of a client.

This embodiment provides a sensing device. The green light chip 2, the red light chip 3, and the infrared light chip 4 of the sensing device are disposed on the front face 11 of the substrate in a shape of a triangle to ensure the light emission concentration of the sensing device and improve the detection accuracy. Six back face pads are disposed on the back face 12 of the substrate. The six back face pads include two common-positive back face pads, three negative back face pads, and an idle back face pad. The back face pads are disposed on the back face 12 of the substrate to facilitate the heat dissipation of the sensing device during working. The heat dissipation efficiency is improved through the two common-positive back face pads. The idle back face pad is disposed to maintain the stability of the sensing device.

In an embodiment, for the manufacturing of a sensing device, an embodiment of the present disclosure provides a manufacturing method of a sensing device. In an embodiment, the combination of all chips on a sensing device is considered as a chipset. Each sensing device needs to be encapsulated on a substrate by an encapsulation material. In practical processing, a single independent sensing device is generally obtained by processing and cutting an entire board.

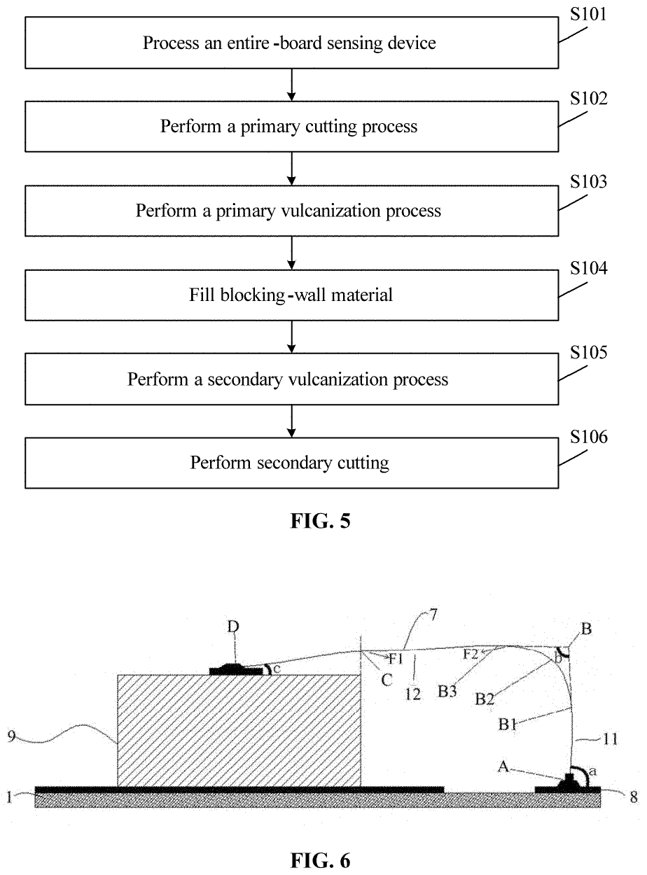

FIG. 5 is a flowchart of a manufacturing method of a sensing device according to an embodiment of the present disclosure.

In an embodiment, the present disclosure provides a manufacturing method of a sensing device. The method is configured to process a sensing device and includes the steps described below.

In S101, an entire-board sensing device is processed.

The entire-board sensing device is obtained by processing based on the structure and quantity of sensing devices. The entire-board sensing device includes a total circuit board, several chipsets, and a total encapsulation layer. The several chipsets are arrayed on the total circuit board and encapsulated by the total encapsulation layer. In an embodiment, the improvement of the manufacturing method of the sensing device in the embodiment of the present disclosure is mainly from the subsequent processing process of the entire-board sensing device defined in the embodiment of the present disclosure. Therefore, the manufacturing method of the sensing device in the embodiment of the present disclosure is described based on the entire-board sensing device structure defined in the embodiment of the present disclosure.

In an embodiment, the processing of the entire-board sensing device is mainly related to processes such as die bonding, wire bonding (soldering), and encapsulation. For specific implementation, reference may be made to the related art, and description is not repeated in embodiments of the present disclosure.

In an implementation, the encapsulation material may be material such as silicone resin, silica gel, or epoxy resin. For a specific use scenario of the sensing device, the encapsulation material used for the entire-board sensing device provided in the embodiment of the present disclosure is silicone resin.

In S102, a primary cutting process is performed.

In an embodiment, by using a dicing saw device, a runner structure is obtained by cutting on the total encapsulation layer through a cutting process. According to the structural feature of the array layout of the sensing devices on the runner in this embodiment of the present disclosure, the obtained runner structure is in a shape of mesh.

In an embodiment, the mesh-shaped runner structure divides the total encapsulation layer into several sub-encapsulation layers (that is, an encapsulation layer corresponding to a single sensing device, and each encapsulation layer is named as a sub-encapsulation layer subsequently for distinction). Any one of the several chipsets is encapsulated by a corresponding one of the several sub-encapsulation layers. One chipset and the sub-encapsulation layer corresponding to the chipset correspond to one sensing device.

In an implementation, to facilitate subsequent spray operations and ensure the forming quality of a sub-side vulcanization-resistant film, the runner structure includes several sub-runners. In the radial section of any one of the several sub-runners, the bottom width of one sub-runner is less than the top width of the sub-runner. In an embodiment, in the radial section of any one of the several sub-runners, the width of one sub-runner gradually increases from bottom to the top of the sub-runner, that is, the sidewall of the sub-runner is a slope. With this arrangement, it can be ensured that the side face of a sub-encapsulation body can be well sprayed with the vulcanization-resistant material, and the forming quality of the sub-side vulcanization-resistant film can be ensured.

In an implementation, correspondingly, to ensure the processing effect of the primary vulcanization process, in the radial section of any one of the several sub-runners, the minimum value of the bottom width of one sub-runner is 0.1 mm, and the minimum value of the difference between the top width of the sub-runner and the bottom width of the sub-runner is 0.03 mm.

In addition, section widths of the runners in the runner structure by primary cutting need to meet requirements of spraying, and requirements of a blocking wall structure obtained by subsequent secondary cutting and avoiding waste of material also need to be considered.

In an embodiment, when the thickness of a required sub-blocking wall is constant, the wider the section width of the runner by primary cutting, the more blocking-wall material needs to be filled, and the more blocking-wall material needs to be cut and discarded. Therefore, considering the preceding factors, in an implementation, the minimum thickness of the sub-blocking wall is 0.1 mm. The section width of the runner is designed according to requirements of the thickness of the sub-blocking wall.

In S103, a primary vulcanization process is performed.

In the runner structure, the outer side face of any one of the several sub-encapsulation layers and the top face of the total circuit board corresponding to the runner structure are sprayed with vulcanization-resistant material through spray process. The vulcanization-resistant material forms a total-side vulcanization-resistant film.

In an embodiment, according to the step of the subsequent secondary cutting process, the step of the secondary cutting process generally requires the entire-board sensing device to be cut along the same dicing trajectory by a narrower dicing saw tool. Therefore, correspondingly, the section width of the runner obtained by the secondary cutting process is narrower, and in the step of the primary cutting process, the section width of the runner obtained by a dicing saw tool is wider.

Correspondingly, to make the spraying of the vulcanization material more convenient, in this embodiment of the present disclosure, the spraying of the vulcanization material is performed in the runner obtained by a primary cutting process to ensure the film-forming covering effect of the vulcanization material.

Due to the limitation of the processing device, in addition to covering outer side faces of the sub-encapsulation layers, the vulcanization-resistant material also covers bottom faces of the runners, that is, the top face of the total circuit board corresponding to the runner structure. Therefore, in addition to covering side faces of the sub-encapsulation layers, the final formed total-side vulcanization-resistant film also covers the bottom face corresponding to the runner structure.

It is to be noted that since the spray range of a nozzle is regional, part of the vulcanization-resistant material is formed on the top face of the total encapsulation layer (sub-encapsulation layers) in actual processing.

In an implementation, the vulcanization-resistant material is silicone resin. The purpose of choosing silicone resin to be used as the vulcanization-resistant material is to keep consistent with the material of the encapsulation material so that the bonding tightness of the vulcanization-resistant material and the encapsulation material can be ensured. In addition, the silicone resin is compact in density and has a better vulcanization-resistant effect.

In S104, blocking-wall material is filled.

The blocking-wall material is filled in the runner structure. The blocking-wall material is solidified in the runner structure to form a total blocking wall. The top face of the total blocking wall is combined with the top face of each sub-encapsulation layer of the several sub-encapsulation layers to form a sprayed top face.

Since the spray process is characterized in that a thin film structure is formed on the surface of an object. Therefore, after the sub-side vulcanization-resistant film is formed, the runner structure is retained. According to structure requirements of the sensing device, in this embodiment of the present disclosure, the blocking-wall material is filled in the runner structure.

Correspondingly, the filling of the blocking-wall material may be achieved by compression molding. The top face of the total blocking wall formed by the solidification of the blocking-wall material is kept flat with the top face of each of the several sub-encapsulation layers, thereby forming a flat sprayed top face.

It is to be noted that for the total blocking wall structure formed by compression molding, there may be part of the blocking-wall material formed on the top face of the total encapsulation layer due to the pressure of the compression molding and the fit between a mold and the total encapsulation layer. Generally, after the total blocking wall structure is formed, the top face of the semi-finished sensing device needs to be ground and processed by grinding or the like to remove the blocking-wall material on the top face of the total encapsulation layer and avoid blocking the light emission of the sensing device. The vulcanization-resistant material formed on the top face of the total encapsulation layer (sub-encapsulation layers) is removed along with the grinding of the blocking-wall material.

In an implementation, the blocking-wall material is a silicone resin material containing titanium dioxide. On the one hand, the blocking-wall material is a silicone resin material to ensure the bonding tightness of the blocking-wall material and the encapsulation material. On the other hand, the titanium dioxide can provide a higher light reflectance and improve the light emission efficiency of the sensing device.

In S105, a secondary vulcanization process is performed.

The vulcanization-resistant material is sprayed on the sprayed top face. The vulcanization-resistant material forms a total top vulcanization-resistant film covering the sprayed top face.

In an embodiment, the sprayed top face is a flat plane. The vulcanization-resistant material is sprayed on the sprayed top face through the spray process. After the vulcanization-resistant material is solidified, a total top vulcanization-resistant film 18 is formed on the sprayed top face.

In S106, secondary cutting is performed.

In an embodiment, the total top vulcanization-resistant film, the total blocking wall and the total circuit board are cut through the cutting process. The total top vulcanization-resistant film is cut into several top vulcanization-resistant films. The total blocking wall is cut into several sub-blocking walls. The total circuit board is cut into several sub-circuit boards (that is, substrates).

After the secondary cutting, the entire-board sensing device is cut into single sensing devices having a specific structure. In a single sensing device, a sub-encapsulation body is wrapped by a sub-top vulcanization-resistant film and a sub-side vulcanization film. The blocking-wall material is formed at the corresponding position.

For a single sensing device obtained from the trajectory of the secondary cutting combining with the cutting, there are always cutting traces on the cutting face of the material. In this embodiment of the present disclosure, the cutting position of the secondary cutting does not affect the specific function of the sensing device, such as the protection of the vulcanization material to the encapsulation material and the restriction of the inner side of the blocking-wall material to the light-emitting angle of a chip.

In summary, the embodiments of the present disclosure provide a sensing device and a manufacturing method of a sensing device. The sensing device includes a substrate and three light-emitting chips. The three light-emitting chips are disposed on the front face of the substrate in a shape of a triangle so that the light emitted by the device is concentrated in the central region, and the monitoring accuracy is improved. The back face pads are disposed on the back face of the substrate so that the heat dissipation efficiency of the device is improved, and the service life of the device is ensured. According to the manufacturing method of a sensing device, the vulcanization-resistant material is sprayed on the runner structure before the runner structure is filled with the blocking-wall material and the vulcanization-resistant material is sprayed on the sprayed top face after the runner structure is filled with the blocking-wall material. Thus, the sub-side vulcanization-resistant film covers the outer side face of the sub-encapsulation layer, the sub-side vulcanization-resistant film extends from the bottom of the outer side face of the sub-encapsulation layer toward the edge of the sub-circuit board and covers the top face of the sub-circuit board, and the sub-top vulcanization-resistant film covers the top face of the sub-encapsulation layer, the top face of the sub-side vulcanization-resistant film, and the top face of the sub-blocking wall. The vulcanization-resistant structure obtained in this implementation has better encapsulation and protection for the sub-encapsulation body and can well avoid the vulcanization of the sub-encapsulation body and the problem of adhesion to the outside. Redundant sub-side vulcanization-resistant film and the sub-top vulcanization-resistant film can increase the intrusion path of impurities and provide good protection to the sub-encapsulation body. In the final step of obtaining the sensing device by cutting and separating, the cutting face cannot have a substantial impact on the function of the sensing device so that the implementation requirements of the cutting operation can be reduced. The sensing device obtained based on the manufacturing method of the sensing device has the characteristics of excellent vulcanization-resistant performance and no external adhesion.

In an embodiment, the preceding manufacturing method of the sensing device is an ideal processing form of the sensing device. In an implementation process, the step of filling the blocking-wall material is generally achieved by a molding method. Due to problems such as the height individual difference of the sensing devices and the matching difference between a mold and a semi-finished product, the blocking-wall material can cover the top face of the semi-finished product after the molding process. To ensure the penetration of the light, the blocking-wall material covering the top face of the semi-finished product needs to be removed.

In an embodiment, in this embodiment of the present disclosure, the technical method for removing excess blocking-wall material is grinding.

In an embodiment, after the blocking-wall material is filled, the semi-finished product is placed on a grinding fixed platform. The grinding work end of a grinding device is controlled to operate. The height of the grinding fixed platform is used as a reference. The grinding work end grinds from the top of the semi-finished product towards the grinding fixed platform and is operated to a preset height.

In an embodiment, for the grinding device, if there is no related feedback setting, the motion drive of the grinding device is substantially independent of a grinding object (that is, the semi-finished product in this embodiment of the present disclosure). Therefore, in this step, after the semi-finished product is placed on the grinding fixed platform, the grinding device is driven to operate according to a set program. The height of the grinding fixed platform is used as a reference. The grinding work end is started and operated to a preset height. By this embodiment, theoretically, the total encapsulation layer and the total blocking wall on the semi-finished product can be ground to the preset height.

To ensure that the actual implementation situation is consistent with the theoretical situation, improvements in various aspects involved in the grinding step need to be made.

In an embodiment, in this embodiment of the present disclosure, the grinding device includes the grinding fixed platform (vacuum chuck), the grinding work end (grinding wheel), and a trimming device (polishing wheel).

In an embodiment, the height of the platform face of the grinding fixed platform, that is, the height of the top face of the vacuum chuck, is constant. In an actual operation, the height of the top face of the vacuum chuck is used as a reference.

In an embodiment, in this embodiment of the present disclosure, considering the convenience of implementation, the grinding work end is a grinding wheel whose grinding face is parallel to the grinding fixed platform. For the manufacturing material of objects to be ground (that is, the total encapsulation layer and the total blocking wall), the grinding wheel is preferably a resin grinding wheel.

Correspondingly, when a grinding wheel is used as the grinding work end, because the surface of the grinding wheel is rough, and when the grinding wheel is a resin grinding wheel, there is a problem of sticking of the grinding wheel during the operation, causing a poor grinding effect and a large roughness; therefore, before each grinding operation and during the grinding process, the grinding face of the grinding wheel needs to be trimmed in time. On the one hand, the sticking material brought out by grinding needs to be removed so that the actual grinding face of the grinding wheel is highly consistent with a theoretical grinding face.

In actual implementation, when the grinding work end, that is, the grinding face of the grinding wheel, is sticky, the grinding efficiency is reduced on the one hand, and on the other hand, the grinding face is uneven, affecting the grinding accuracy. Therefore, in the grinding step, the grinding work end is ground from the top of the semi-finished product toward the grinding fixed platform and operated to a preset height through several sub-steps. In each sub-step, the grinding face of the grinding work end is operated to a corresponding theoretical height. In the last performed sub-step, the grinding face of the grinding work end is operated to a preset height. That is, the overall grinding process needs to be achieved by several independent grinding operations. For each independent grinding operation, to ensure the grinding accuracy, the manner described below can be implemented.

In an embodiment, each sub-step includes the steps described below.

Adjustment of the trimming device: the height of the platform face of the grinding fixed platform is used as a reference, and the height of the trimming face of the trimming device is the same as a theoretical height corresponding to the sub-step. In an embodiment, the polishing face of the trimming device is consistent with the polishing height of the semi-finished product required by the grinding device in the sub-step to ensure that the polishing height of the grinding surface of the grinding wheel is consistent with the grinding height corresponding to the grinding face of the grinding wheel in the sub-step to provide a better grinding effect.

Grinding of the grinding device: after the grinding device is driven to operate above the trimming device located on the outside of the grinding fixed platform, the grinding device is driven to work, and the grinding face of the grinding device is operated to a corresponding theoretical height. The grinding face coincides with the trimming face. This step makes the grinding face coincide with the trimming face, that is, the grinding face is consistent with the corresponding theoretical height.

In-feed grinding: the grinding device is driven to translate above the grinding fixed platform to grind the semi-finished product.

It is to be noted that, when necessary, the grinding of the grinding face of the grinding wheel can run through the entire process flow of the sensing device. The reasonable timing of the grinding of the grinding wheel is determined through empirical judgment, statistical calculation, visual observation, and the like so that the grinding accuracy and the grinding speed of the grinding wheel are taken into account, thereby ensuring the high efficiency of the grinding operation.

In an embodiment, the grinding wheel can be worn out with use, resulting in a change in the grinding face. Theoretically, the grinding operation can be completed by the presence of at least one grinding particle on the grinding face. Correspondingly, if no grinding particle is present on the grinding face, it indicates that the grinding face fails, and adjustment of the grinding face is required.

In an implementation of this embodiment of the present disclosure, whether the width of the gap between the trimming device and the grinding wheel being greater than a preset value is observed based on a visual device, and if the width of the gap between the trimming device and the grinding wheel is greater than the preset value, the grinding wheel is controlled to move the minimum stepping downward. The height of the grinding wheel moving the minimum stepping downward is taken as a theoretical grinding height.

In an embodiment, if the gap is greater than the preset value, that is, the original grinding face fails, the grinding face needs to be adjusted to keep an actual grinding face consistent with a theoretical grinding face.

In an embodiment, the minimum stepping in the case where the height of the grinding wheel moving the minimum stepping downward is taken as a theoretical grinding height refers to the minimum stepping of the movement of the grinding wheel driven by an external device along a vertical direction. According to this implementation method, the grinding face of the grinding wheel can be adjusted in real time to avoid inaccurate height of the semi-finished product obtained by grinding.

In an embodiment, for requirements of the roughness to be ground, the value range of the mesh number of the grinding wheels is [500, 1000].

Correspondingly, to ensure the grinding effect, the value range of the rotational speed of the grinding wheel in the operation state is [600 rpm, 800 rpm].

Correspondingly, under a corresponding rotational speed, to take into account the grinding efficiency and the grinding effect, the value range of the in-feed speed of the grinding wheel which grinds from the top of the semi-finished product towards the grinding fixed platform and is operated to a preset height is [0.1 μm/s, 0.3 μm/s].

Correspondingly, to ensure that the grinding effect meets the requirements, the top face of the semi-finished product after grinding needs to be performed roughness detection. When the roughness of the top face of the semi-finished product after grinding is less than or equal to 0.5, the secondary cutting step is performed. When the roughness of the top face of the semi-finished product after grinding is greater than 0.5, the grinding step is performed again.

Correspondingly, the preceding implementation is mainly used for adjusting the implementation structure of a grinding wheel. In an implementation, for the semi-finished product, the grinding fixed platform is a chuck. The chuck can ensure the adsorption of the bottom face of the total circuit board on the semi-finished product so that the bottom face of the total circuit board is tightly attached to the chuck, thereby ensuring that the height of the bottom face of the semi-finished product remains the same as the height of the top face of the chuck. The height of the top face of the chuck may be expressed as the height of the bottom face of the semi-finished product. The height of the grinding wheel is based on the height of the top face of the chuck, ensuring that the height of the semi-finished product after grinding is the same as a theory value.

FIG. 6 is a sectional view of a sensing device according to an embodiment of the present disclosure. Referring to FIG. 6, the sensing device includes a metal wire 7 and an encapsulation layer. The substrate has a pad 8, that is, the preceding front face pad. LED chip 9 (for example, a red light chip) is fixed on the substrate 1 and located on a side of the pad 8. At least one LED chip 9 has a top electrode. The top electrode is electrically connected to the pad 8 by the metal wire 7. The LED chip 9 and the metal wire 7 are encapsulated by the encapsulation layer.

In an embodiment, the encapsulation layer is used for encapsulating LED chips and connecting lines. The height of the chip on the sensing device is h. The height of the encapsulation layer 10 is H. The constraint relationship between H and h is 1.5 h<H<4 h. The value range of H is 200 μm<H<600 μm. To avoid affecting the showing of the view, these are not shown in FIG. 6.

The metal wire includes a first segment AB3, a second segment B3C and a third segment CD connected in sequence.

In an embodiment, the head end of the first segment AB3 is bonded on the pad 8. The included angle, facing away from the side of the LED chip, between the first segment AB3 and the top face of the pad 8 is a. The middle portion of the first segment AB3 has a first camber arch 11 facing away from the LED chip 9. Since a wire nozzle is always facing downward, after the wire nozzle fuse the head end of the metal wire 7 into a gold ball, the direction of the metal wire 7 taken out of the gold ball is close to being perpendicular to the top face of the pad 8. In this embodiment of the present disclosure, the driving of the wire nozzle forms the required trajectory of the metal wire 7. According to the design of this embodiment of the present disclosure, a first camber arch 11 is formed in the first segment AB3. The first camber arch 11 is recessed in the direction away from the LED chip 9. In an embodiment, the forming mechanism and the effect of the first camber arch 11 are described combined with the overall structure of the metal wire 7 sub sequently.

In an embodiment, the transition between the first segment AB3 and the second segment B3C is a rounded corner B1-B3. The included angle, facing the LED chip, between the first segment AB3 and the second segment B3C is b. In an embodiment, the transition between the first segment AB3 and the second segment B3C is the rounded corner B1-B3. Therefore, in actual implementation, the bending point shown at point B is not generated. The connecting rounded corner between the first segment AB3 and the second segment B3C sequentially passes through point B1, point B2, and point B3.

In an embodiment, the middle portion of the second segment B3C has a second camber arch 12 facing downwardly. The connection position between the second segment B3C and the third segment CD is located directly above the edge of the top face of the LED chip. In an embodiment, two ends of the second segment B3C are substantially metal wires connected to the tail end of the first segment AB3 and the head end of the third segment CD. The tension between the first segment AB3 and the third segment CD may be adjusted by adjusting the length of the second segment B3C. In this embodiment of the present disclosure, to avoid excessive internal stress between the first segment AB3 and the gold ball, the length of the second segment B3C should not be too short to avoid pulling the first segment AB3. Correspondingly, to avoid the connection position between the third segment CD and the second segment B3C being too close to the top face of the LED chip, the length of the second segment B3C needs to be avoided being too long to prevent the weight of the second segment B3C from being heavier and lowering the height of the head end of the third segment CD. At the same time, it is avoided that the length of the second segment B3C is too long to cause the height of the lowermost end of the second camber arch 12 to be too low. In the specific implementation, there is no quantified limit data for the specific length of the second segment B3C under the premise of ensuring the height of point C. However, in this embodiment of the present disclosure, due to the position limitation of point C located directly above the edge of the top face of the LED chip 9, the distance limitation between point C and the top face of the LED chip 9, and the angle limitation of a, in actual implementation, the length of the second segment B3C is correspondingly limited. If the length of the second segment B3C is too small, the angle of a does not meet the limit requirements. If the length of the second segment B3C is too long, the supporting force of the third segment CD is insufficient, and point C is depressed.

In an embodiment, the tail end of the third segment CD is bonded on the top electrode. The included angle, facing the LED chip, between the third segment CD and the top face of the top electrode is c. In an embodiment, since the wire nozzle needs to cut the metal wire on the solder ball of the top electrode. The direction of the tangent line and the orientation of the wire nozzle are close to vertical. Therefore, the included angle c between the metal wire on the top electrode and the top electrode is a small included angle. After the bonded material is solidified, the tail end of the third segment CD is equivalent to being fixed on the top electrode. Therefore, the third segment CD is actually fixed by the solder ball of the top electrode.

In conjunction with the preceding structure of the metal wire, the head end of the first segment AB3 is fixed on the gold ball of the pad 8. The tail end of the first segment AB3 is located above the head end of the first segment AB3. The first segment AB3 can be regarded as a soft rod structure with one end fixed. The tail end of the third segment CD is fixed on the solder ball of the top electrode. The head end of the third segment CD is located directly above the edge of the top face of the top electrode. The third segment CD can be regarded as a soft rod with one end fixed on the solder ball. The second segment B3C can be regarded as a soft rod structure of which two ends are connected to the tail end of the first segment AB3 and the head end of the third segment CD.

Since the head end of the first segment AB3 is very firmly bonded on the pad, and the pad has a strong fixing effect on the head end of the first segment AB3, the head end of the first segment AB3 may be regarded as a fixed morphological structure. Similarly, the bonding between the tail end of the third segment CD and the top electrode is very firm and reliable, and the top electrode has a strong fixing effect on the tail end of the third segment CD. Therefore, the tail end of the third segment CD can be regarded as a fixed morphological structure. Correspondingly, attitude changes of other positions of the first segment AB3 and attitude changes of other positions of the third segment CD are related to the second segment B3C.

The acting forces generated by the second segment B3C on the third segment CD and the first segment AB3 are F1 and F2, respectively (the angle of an acting force is actually the direction of a tangent line, and because the angle of the tangent line is too small to be indicated, a similar direction is used for identification in FIG. 6). First, the wire nozzle is controlled by the trajectory of the metal wire to generate the rounded corner B1-B3. Then the head end of the second segment B3C is connected at point B3. The second segment B3C is involved in the bending of the rounded corner B1-B3 at point B3. Correspondingly, since the first segment AB3 is not a hard rod, but a soft rod, the first camber arch 11 is formed at the first segment AB3. Since the existence of the first camber arch 11 and the rounded corner B1-B3, the acting force generated by the bending of the first camber arch 11 is used to support the head end of the second segment B3C at point B3. Then, since the length of the third segment CD is short, the head end of the third segment CD is maintained at point C by the fixing acting force of the tail end and the top electrode. When the weight of the second segment B3C does not exceed a preset range, the position of point C is hardly changed.

In the actual situation, according to the device structure provided in this embodiment of the present disclosure, the starting point of the BSOB wire bonding process is set on the pad due to the height difference between the pad and the top electrode during processing so that the front section of the metal wire 7 that is close to the vertical can maintain a certain length margin, thereby avoiding excessive bonding internal stress between the metal wire 7 and the pad. Correspondingly, the reduction of the arc height of the metal wire 7 does not have a great influence on the bonding internal stress between the metal wire 7 and the pad, thereby ensuring the stability of the structure of the metal wire 7. The end point of the BSOB wire bonding process is set on the top electrode. The end section of the metal wire 7 located in the outline surrounding area of the LED chip 9 can be well fixed by the solder ball on the top electrode, preventing the connection position between the second segment B3C and the third segment CD from being in contact with the LED chip.

In an embodiment, according to actual implementation statistics, the height of the LED chip 9 is h, the height (that is, the height from the highest point B3 to the top face of the substrate) of the highest point (point B3 shown in FIG. 6) of the metal wire 7 is less than or equal to 1.5 h, and the height of the highest point of the metal wire is greater than h. In an embodiment, the height of the middle point B2 of the rounded corner between the first segment AB3 and the second segment B3C is greater than or equal to the height of a light-emitting chip 3. By defining the height of point B2, when the substrate is deformed, the height of point B3 can be prevented from falling below a threshold, resulting in contact between the second camber arch 12 and the side face of the LED chip.

Correspondingly, to leave an appropriate position margin, the height of the LED chip is h, and the maximum value of the height (that is, the height of point B3) of the highest point of the rounded corner at the connection position between the first segment and the second segment is 1.5 times the height of the light-emitting chip.

In an embodiment, to leave an appropriate position margin, the height from the connection position between the second segment B3C and the third segment CD to the top face of the chip is greater than the wire diameter of the metal wire.

In an embodiment, the wire diameter of the metal wire is 25 μm. Correspondingly, the height from the connection position between the second segment B3C and the third segment CD to the top face of the chip is greater than 25 μm.

Further, the height from the connection position between the second segment B3C and the third segment CD to the top face of the chip is less than or equal to h/2, and the height from the connection position between the second segment and the third segment to the top face of the LED chip is greater than or equal to h/6. In this setting, it can be ensured that when the substrate is thermally deformed, the contact probability between the connecting wires and the chips is reduced.

In an embodiment, since the bonding position of the top electrode needs to generate sufficient supporting force for the third segment CD, the bonding strength of the tail end of the third segment CD to the top electrode is not high due to the third segment CD cut by the wire nozzle during conventional processing. Therefore, to ensure the shape stability of the third segment CD, and ensure that the third segment CD can provide sufficient supporting capability at point C, the bonding position between the tail end of the third segment CD and the top electrode is processed with a soldered gold ball.

Similarly, to increase the supporting capability of the first segment AB3 at point B1, the bonding position between the first segment AB3 and pad is processed with a soldered gold ball.

In an embodiment, the value range of the rounded corner at the connection position between the first segment AB3 and the second segment B3C is [80°, 100° ]. If the value range of the rounded corner is too small, the structure of the metal wire is unstable. If the value range of the rounded corner is too large, the absorption capability of the metal wire to the stress is deteriorated, and the impact resistance of the metal wire is reduced.

Further, the distance between the chip and the pad is 90 μm to 1050 μm. In an embodiment, the distance between the chip and the pad refers to the distance between the edge of the chip toward the pad and the edge of the pad toward the chip. In an embodiment, there is a need for sufficient distance space to form the connecting wire structure required in this embodiment of the present disclosure. In this case, the risk of line breakage due to excessive connecting wire span needs to be avoided.

In an embodiment, the height of the chip is greater than 100 μm and less than 350 μm. The distance between the chip and the pad is greater than 0.9 times the height of the chip and less than three times the height of the chip. Correspondingly, it is estimated that the distance between the chip and the pad is 90 μm to 1050 μm.

Further, the value range of the included angle a is 80°<a<110° to ensure that the lateral component of the first segment is not too large, thereby ensuring the supporting stability of the first segment.

Further, the value range of the included angle c is 8°<c<25° to ensure that the lifting height of the connecting line from point D to point C and from point C to point B3 is low, thereby facilitating the lowering of the height of the encapsulation layer.

Further, in an actual operation, if the LED chip has two top electrodes, considering the symmetry and the increase of the risk of line collapse caused by the distance from the two top electrodes to the edge of the corresponding LED chip being too large, correspondingly, the width of the LED chip is 2 to 2.5 times the distance between the chip electrode and the edge of the chip. That is, the chip electrode is located in the central region of the top face of the LED chip.

Further, the value range of the included angle d between the second segment and the horizontal plane is 8°<d<25°. This angle is generally associated with the angle of the included angle c. The limitation of the angle d can ensure that the final height of B3 is low.

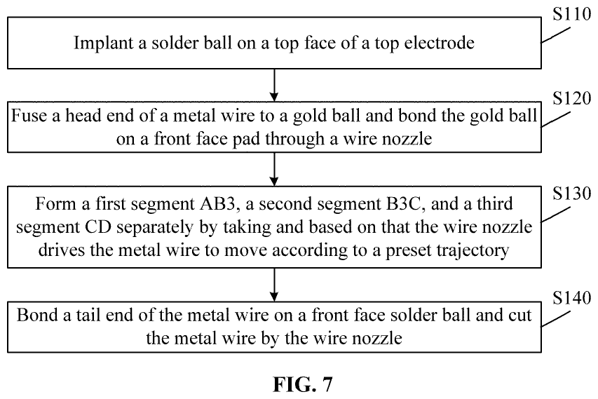

FIG. 7 is a flowchart of a wire bonding method according to an embodiment of the present disclosure.

Correspondingly, the present disclosure further provides a wire bonding method. The method is used for the wire bonding of a metal wire between a top electrode and a corresponding front face pad in a sensing device. The method includes the steps described below.

In S110, a solder ball is implanted on a top face of a top electrode.

The top electrode serves as the processing end point of the BSOB process. To avoid damage to the top electrode, it is necessary to implant a solder ball on the top face of a top electrode for soldering.

In S120, a head end of a metal wire is fused to a gold ball and bonded on a front face pad through a wire nozzle.

At the beginning of the BSOB process, the head end of the metal wire is fused to the gold ball and bonded on the front face pad by using a wire nozzle. After the gold ball is solidified, the gold ball is connected to and fixed on the front face pad.

In S130, a first segment AB3, a second segment B3C, and a third segment CD are separately formed by taking and based on that the wire nozzle drives the metal wire to move according to a preset trajectory.

By the simultaneous movement of the taking of the metal wire and the wire nozzle, the metal wire is moved according to the preset trajectory to finally form the desired structure of the first segment AB3, the second segment B3C, and the third segment CD.

In S140, a tail end of the metal wire is bonded on a front face solder ball and cut by the wire nozzle.

The end of the wire nozzle falls on the solder ball. The metal wire is cut by fusing the metal wire and physically cutting to form metal wires.

According to the wire bonding method provided in this embodiment of the present disclosure, the starting point of the BSOB process is set on a front face pad so that the arc height can be reduced.

In view of the above, according to the wire bonding method of the device provided in this embodiment of the present disclosure, the starting point of the BSOB wire bonding process is set on the pad due to the height difference between the front face pad and the top electrode during processing so that the front section of the metal wire that is close to the vertical can maintain a certain length margin, thereby avoiding excessive bonding internal stress between the metal wire and the front face pad. Correspondingly, the reduction of the arc height of the metal wire does not have a great influence on the bonding internal stress between the metal wire and the front face pad, thereby ensuring the stability of the structure of the metal wire. The end point of the BSOB wire bonding process is set on the top electrode. The end section of the metal wire located in the outline surrounding area of the LED chip can be well fixed by the solder ball on the top electrode, preventing the connection position between the second segment and the third segment from being in contact with the LED chip. When the substrate is deformed by heat, due to the existence of the first camber arch, the rounded corner, and the second camber arch, the rounded corner swings towards the position away from the center of a circle when the substrate is deformed. The second camber arch is subjected to upward stress. The depression of the camber arch buffers the stress. The depression gradually becomes gentle. The overall height of the metal wire does not change greatly in the process of adapting to the deformation of the substrate, thereby ensuring that the metal wire does not touch an encapsulation mold and ensuring the smoothness forming of the encapsulation layer and the smooth processing of the device.

It is to be noted that the preceding are only preferred embodiments of the present disclosure and technical principles used therein. It is appreciated by those skilled in the art that the present disclosure is not limited to the embodiments described herein. Those skilled in the art can make various apparent modifications, adaptations, combinations, and substitutions without departing from the scope of the present disclosure. Therefore, while the present disclosure has been described in detail through the preceding embodiments, the present disclosure is not limited to the preceding embodiments and may include more other equivalent embodiments without departing from the concept of the present disclosure. The scope of the present disclosure is determined by the scope of the appended claims.

What is claimed is:

1. A sensing device, comprising:
a substrate, a red light chip, an infrared light chip, and a green light chip, wherein the red light chip, the infrared light chip, and the green light chip are disposed on a front face of the substrate;
wherein five front face pads are disposed on the front face of the substrate, wherein the five front face pads comprise a first front face pad, a second front face pad, a third front face pad, a fourth front face pad, and a fifth front face pad;
wherein five back face pads are disposed on a back face of the substrate, wherein the five back face pads comprise a first back face pad, a second back face pad, a third back face pad, a fourth back face pad, and a fifth back face pad, and the third back face pad is connected to the fourth back face pad by a conductive line;
wherein one of the five front face pads is electrically connected to a corresponding one of the five back face pads; and
wherein the red light chip, the infrared light chip, and the green light chip are electrically connected to the five front face pads.

2. The sensing device according to claim 1, wherein
a bottom electrode of the green light chip is bonded on the second front face pad, and a top electrode of the green light chip is electrically connected to the third front face pad by a metal wire;
a bottom electrode of the red light chip is bonded on the fourth front face pad, and a top electrode of the red light chip is electrically connected to the fifth front face pad by a metal wire; and
a bottom electrode of the infrared light chip is bonded on the fourth front face pad, and a top electrode of the infrared light chip is electrically connected to the first front face pad by a metal wire.

3. The sensing device according to claim 1, wherein each of a size of the red light chip and a size of the infrared light chip is less than a size of the green light chip.

4. The sensing device according to claim 1, wherein the red light chip, the infrared light chip, and the green light chip are disposed on the front face of the substrate in a shape of a triangle; the red light chip and the infrared light chip are disposed on a same side; and the green light chip is disposed on another side.

5. The sensing device according to claim 1, wherein the front face of the substrate is provided with an encapsulation layer formed by an encapsulation material, and the encapsulation layer is surrounded by a white blocking wall.

6. The sensing device according to claim 5, wherein a light-emitting angle of the sensing device is α, and a constraint relationship of α is 120°≤α≤130°.

7. The sensing device according to claim 1, wherein a sixth front face pad further is disposed on the front face of the substrate, or a sixth back face pad is further disposed on the back face of the substrate, or a sixth front face pad is further disposed on the front face of the substrate and a sixth back face pad is further disposed on the back face of the substrate, wherein the sixth front face pad is an idle front face pad, and the sixth back face pad is an idle back face pad.

8. The sensing device according to claim 7, wherein the first back face pad, the second back face pad, the third back face pad, the fourth back face pad, the fifth back face pad, and the sixth back face pad are separated into two columns and disposed on the back face of the substrate; and the third back face pad and the fourth back face pad are disposed at diagonal positions.

9. The sensing device according to claim 8, wherein the third back face pad and the fourth back face pad are common-positive back face pads; the first back face pad, the second back face pad, and the fifth back face pad are negative back face pads; and the sixth back face pad is the idle back face pad; or
the third back face pad and the fourth back face pad are common-negative back face pads; the first back face pad, the second back face pad, and the fifth back face pad are positive back face pads; and the sixth back face pad is the idle back face pad.

10. The sensing device according to claim 8, wherein the conductive line comprises three segments, and an included angle between two adjacent segments of the three segments is a right angle.

11. The sensing device according to claim 10, wherein the back face of the substrate is coated with green oil, and a middle segment of the three segments is close to one column of the two columns in which the first back face pad, the second back face pad, the third back face pad, the fourth back face pad, the fifth back face pad, and the sixth back face pad are disposed; and a position between the middle segment of the three segments and another one column of the two columns in which the first back face pad, the second back face pad, the third back face pad, the fourth back face pad, the fifth back face pad, and the sixth back face pad are disposed is blank to form an electrical mark.

12. The sensing device according to claim 10, wherein a thickness of the conductive line is less than a thickness of one of the first back face pad, the second back face pad, the third back face pad, the fourth back face pad, the fifth back face pad, or the sixth back face pad.

13. The sensing device according to claim 1, further comprising:
a metal wire and an encapsulation layer, wherein the red light chip, the infrared light chip, and the green light chip are light-emitting diode (LED) chips, each of at least one LED chip of the LED chips has a top electrode, the top electrode is electrically connected to one front face pad by the metal wire, and the LED chip and the metal wire are encapsulated by the encapsulation layer;
wherein the metal wire comprises a first segment, a second segment, and a third segment connected in sequence;
wherein a head end of the first segment is bonded on the one front face pad, and an included angle, facing away from the LED chip, between the first segment and a top face of the one front face pad is a;
wherein a transition between the first segment and the second segment is a rounded corner, and an included angle, facing the LED chip, between the first segment and the second segment is b;
wherein a connection position between the second segment and the third segment is located directly above an edge of a top face of the LED chip; and
wherein a tail end of the third segment is bonded on the top electrode, and an included angle, facing the LED chip, between the third segment and a top face of the top electrode is c.

14. The sensing device according to claim 13, wherein a middle portion of the first segment has a first camber arch facing away from the LED chip.

15. The sensing device according to claim 13, wherein a middle portion of the second segment has a second camber arch facing a downward direction.

16. The sensing device according to claim 13, wherein a height of the LED chip is h, a height of a highest point of the metal wire is less than or equal to 1.5 h, and the height of the highest point of the metal wire is greater than h.

17. The sensing device according to claim 13, wherein a distance between the connection position between the second segment and the third segment and the top face of the LED chip is greater than a wire diameter of the metal wire.

18. The sensing device according to claim 17, wherein a height of the LED chip is h, the distance between the connection position between the second segment and the third segment and the top face of the LED chip is less than or equal to h/2, and the distance between the connection position between the second segment and the third segment and the top face of the LED chip is greater than or equal to h/6.

19. The sensing device according to claim 13, wherein a value range of a rounded corner of a connection position between the first segment and the second segment is [80°, 100° ], and a distance between the LED chip and the one front face pad is from 90 μm to 1050 μm; and
a value range of the included angle a is 80°<a<110°, a value range of the included angle c is 8°<c<25°, a width of the LED chip is 2 to 2.5 times a distance from the top electrode of the LED chip to an edge of the LED chip, and a value range of an included angle d between the second segment and a horizontal plane is 8°<d<25°.

20. A manufacturing method of a sensing device, configured to manufacture the sensing device according to claim 1.
